(19) United States
(12) Patent Application Publication
(10) Pub. No.: US 2016/0235372 A1
(43) Pub. Date: Aug. 18, 2016

(54) PORTABLE CANCER DIAGNOSTIC DEVICE AND SYSTEM

(71) Applicant: Telebyte, Inc., Hauppauge, NY (US)

(72) Inventors: Kenneth S. Schneider, Hauppauge, NY (US); Joel Strom, Hauppauge, NY (US); Michael Breneisen, Hauppauge, NY (US)

(21) Appl. No.: 14/624,190

(22) Filed: Feb. 17, 2015

(51) Int. Cl. A61B 5/00; A61B 8/08

(52) U.S. Cl. CPC A61B 5/7264; A61B 5/0075; A61B 5/7278; A61B 5/7228; A61B 5/743; A61B 5/0022; A61B 8/085; A61B 8/5223

(57) ABSTRACT

A portable device and system, based upon Diffuse Optical Spectroscopy (DOS), for the detection of surface detectable cancers such as breast cancer and the determination of their response to therapy. The system may include hardware and software components that form a number of subsystems: an Optical-Electronic Subsystem, a Digitization Subsystem, an Optical Parameter Computation Subsystem, an Artificial Intelligence Subsystem, and a Presentation Subsystem. The system can be integrated into a hybrid architecture that utilizes other imaging techniques, such as X-ray mammography, for cancer detection.

### FIELD OF THE INVENTION

The present invention relates to a device and a system for the detection of surface detectable cancers such as breast cancer and the assessment of their response to therapies.

### BACKGROUND INFORMATION

Cancer is a major cause of morbidity and mortality in the United States, and will be more so with the aging of the population. Early detection and classification of malignant tumors combined with accurate assessment of their response to therapy could have important implications, not only for patient care, but also from the public health and economic perspectives. Of particular interest, because of their high incidence, are those tumors that arise close to the body surface. These include, but are not limited to, breast and skin cancers, e.g., squamous cell carcinoma, malignant melanomas and certain throat and neck cancers. Treatment often involves multimodality therapeutics, including radio-, chemo- and immuno-therapies. The immediate concerns of the treating physician are to identify correctly and diagnose a tumor in order to commence targeted therapy and rapidly to determine the patient's therapeutic response.

A number of techniques are currently employed to screen patients for the above mentioned types of tumors. For example, breast cancer screening employs mammography, magnetic resonance imaging (MRI), ultrasonography, thermography and irradiation by microwaves. However, mammography requires compression of the breast which is often uncomfortable for the patient. Mammography also exposes the patient to ionizing radiation and may fail to detect malignant tumors in some patients, especially younger individuals, e.g., those under fifty years old. Most guidelines do not recommend routine screening for these younger patients because of concerns regarding the effects of radiation exposure and false positive identification rates. MRI requires an intravenous contrast injection or that the patient be inconveniently confined in an enclosed space. Also, mammography, MRI, ultrasonography and irradiation typically cannot be performed in a primary care physician's office. Accordingly, a point-of-care device that could be used to screen patients for breast cancer and other surface malignancies and which can assess the effects of therapy would be an important addition to the armamentarium for these diseases.

Diffuse Optical Spectroscopy (DOS) is a proposed method to determine the presence of certain tumor types (such as breast cancer and certain throat cancers) and their response to therapy. DOS is applicable when the suspected cancerous tissue is close to the surface of the body, typically within several centimeters of the surface. DOS involves irradiating a region of the body containing a suspicious or confirmed tumor with coherent light in the Near-infrared (NIR) spectrum by a number of lasers, each emitting a different wavelength or equivalently, each at a different center frequency. Each laser beam is amplitude modulated with a modulation waveform of a certain bandwidth. Upon reaching the surface of the body, part of the light is absorbed into the body by diffusion and the remainder is back-scattered, similar to how a radar signal is reflected off a physical object. The received back-scattered light is processed, e.g., demodulated, and a Fourier spectrum of the resulting processed waveform is computed. This computed spectrum is then used to estimate the corresponding optical properties, namely absorption and scattering parameters. Because cancerous tissue contains a number of physical and chemical differences from non-cancerous tissue, the optical properties can provide a “signature” that can potentially be used to discriminate non-cancerous from cancerous tissue.

Example embodiments of the present invention will be described which use DOS. PhotoAcoustic Imaging (PAI) combines the irradiating part of DOS with ultrasound imaging and molecular targeted contrast agents. The detection part of PAI though is fundamentally different in that it is centered on processing pressure waves rather than electromagnetic waves. PAI provides the capability of imaging cancer at the cellular and molecular level to improve diagnosis of tumors, detection of circulating tumors, metastatic lymph nodes and metastatic melanoma cells. While PAI differs from DOS, parts of the invention described below are directly applicable to PAI. These include, for example, Wavelength Division Multiplexing of the radiation and the use of Artificial Intelligence techniques for the detection and classification of tumors. Along this same line, the innovations in the invention being presented are independent of the specific interrogating imaging modality and can be integrated with other advanced analysis techniques. To the point, they can be applied to multiple imaging methodologies, by way of example shear or strain imaging. Thus, an example embodiment of the present invention involves analyzing data obtained from multiple imaging techniques, such as DOS together with a second imaging technique that involves irradiation with acoustics (e.g., shear or strain imaging) and/or detection of emitted acoustics (e.g., PAI), in order to render a decision using Artificial Intelligence.

FIG. 1 shows an example spectrum of an incident NIR beam at one particular laser NIR center frequency, f_{L}. This could have been equivalently shown as a function of the wavelength, λ with the laser wavelength denoted as λ_{L}. FIG. 2 is an illustrative graph of the magnitude of the Fourier spectra of received scattered demodulated waveforms from both normal tissue and cancerous tissue. FIG. 3 is an illustrative graph of the phase of Fourier spectra of received scattered demodulated waveforms from both normal tissue and cancerous tissue.

FIG. 4A shows example spectra of a plurality of incident laser beams. To show the equivalence of the presentation by either frequency or wavelength this is shown as a function of λ. FIGS. 4B and 4C show corresponding Absorption and Scattering properties, as functions of the wavelength, λ. These spectral graphs are for illustrative purposes only. The curves in FIGS. 4B and 4C are hypothetical and ideally shaped. The responses are depicted for both normal and cancerous tissues. The Fourier spectra are shown as functions of frequency, f. The optical properties are shown as a function of the wavelength, λ, of the incident NIR coherent light. In these and all subsequent figures the units of λ are in nm. As shown in FIGS. 4B and 4C, Absorption and Scattering are typically higher for certain cancerous tissues, such as breast cancer, than for normal tissue, though as indicated in FIG. 4C this may not be true over the full wavelength range. These differences can be then used as a discriminant for cancer detection.

At present, there are a number of justifiable concerns regarding clinical application of DOS:

First, referring to FIG. 4A, while a number of different modulated lasers have been used in previous efforts, the demodulation processing has only provided partial spectral information for use in response determination due to the limitation of the bandwidth of the modulating waveform. This is illustrated in FIGS. 2 and 3. Partial Fourier spectral information, i.e. discontinuous Fourier spectral information with gaps 15/17, leads to gaps 25/27 in corresponding measurements of the optical parameters, Absorption and Scattering, as shown in FIGS. 4B and 4C. The reason for the gaps 15/17 is that the incident modulated laser beams can only generate Fourier spectral information in the modulation bandwidth around the laser carrier. The gaps 15/17 in the Fourier spectral information collected are significant because the resulting gaps 25/27 in the measurements of optical parameters can affect the sensitivity and specificity, and thus accuracy, of the information provided. Specifically, the gaps 25/27 can lead to distortions in the signals that will affect the interpretability with respect to tissue discrimination and thus the accuracy of diagnosis which can result in both false positive and negative diagnoses, both of which can seriously and deleteriously impact patient care. Traditional DOS processing assumes that these gaps contain no information and therefore ignores the gaps. However, this is a very narrow view based upon speculation and a desire to simplify the processing, and it is not necessarily true.

Second, the computation of all of the Fourier spectral information has to be carried out with extreme accuracy. The greater the accuracy in Fourier spectral information, the greater the reliability in the computation of the optical parameters, Absorption and Scattering. This enhances the discrimination of the measured Absorption and Scattering parameters of the normal tissue from the absorption and scattering parameters of the suspected cancerous tissues, which in turn enhances both the reliability of detection of cancerous tissue and determination of the success of the therapeutic program. However, traditional DOS processing ignores the effects of noise, interference, inaccuracies in digitization, biasing in sampling and other deleterious effects.

Third, there is the need to make the device or system small enough to be portable so that it can be employed in the physician's office, rather than sending the patient to a series of appointments at centralized laboratories. To date, attention has not been directed at this concern.

Fourth, the optical parameters, the Absorption and Scattering, should to be computationally processed in such a way as to give the physician a transparent and understandable indication of the detection of cancer, and if so detected, an indication of the patient's response to a subsequent course of therapy. Presenting the optical parameters by themselves to the physician is not worthwhile. To date, attention has not been directed at this concern.

### SUMMARY

Example embodiments of the present invention relate to a portable cancer diagnostic system and device that improves upon traditional DOS approaches and addresses all of the above mentioned concerns regarding clinical application of DOS.

Regarding the issue of gaps in the spectral information, the spectral information and the resulting optical parameters are the result of the interaction between a radiated signal and tissue from a human patient. This presents certain constraints on the behavior of the spectral information and the resulting optical parameters with respect to “smoothness”—basically continuity and the continuity of its mathematical derivatives, e.g., a first derivative of an Absorption curve. Example embodiments of the present invention will be described which take these constraints into account in order to fill in the gaps, thereby deriving extra information that is potentially important in the clinical decision process.

In an example embodiment, the diagnostic device estimates Absorption and Scattering over the full range of wavelengths, λ's, emitted by the lasers in order to achieve the highest possible contrast in the measured Absorption and Scattering parameters, between normal and suspected cancerous tissue. This optimizes the accuracy and reliability of tumor discrimination and the prediction and assessment of the response of the patient's tumor to therapy.

In an example embodiment, the diagnostic device is operated after an initial detection has been confirmed by tissue diagnosis (e.g., biopsy), and after a program of therapy has commenced to determine quickly if the therapy is effective. The initial detection may, but need not, be performed by the same diagnostic device. The irradiated section of the body should ideally contain normal tissue adjacent to tissue from a cancerous lesion, resulting in the computation of two sets of each of the optical parameters, Absorption and Scattering. If a patient is responding to therapy, then it is expected that the measured optical parameters corresponding to the cancerous lesion should move closer to, and ideally coincide with, the optical parameters corresponding to normal tissue as compared to a corresponding set of optical parameters obtained earlier in time, e.g., when the cancerous lesion was initially detected. However, this is not absolutely certain and there are probabilities associated with the resulting physician diagnosis as to whether a patient is responding to the therapy. If the same diagnostic device is used throughout the therapy, the diagnostic device may take a number of measurements of the optical parameters over time, e.g., intervals of weeks or possibly months, in order to assess the patient's progress.

Regarding the issue of portability, most if not all of the system can be integrated into a single diagnostic device. The system can be implemented in a space-efficient manner so that the device can be conveniently placed in the physician's office. In one embodiment, the system includes, in addition to the diagnostic device, processing components that can also be conveniently placed in the physician's office, e.g., a personal computer. In another embodiment, the system includes remote processing components that are accessed, for example, over the Internet. The system can be portable so that the physician can transport it easily from one examining room to another. This shortens the initial cancer detection process, and if cancer is detected and confirmed by biopsy, enables quick determination of whether a program of therapy was successful. Thus, a portable diagnostic device according to an embodiment of the present invention facilitates greater efficiency in physician-patient interaction. Further, by obviating the need to visit outside laboratories for testing, the portable diagnostic device has a much more positive impact on the experience for the patient in this stressful situation. Example embodiments are directed to optical-electronic elements and computational elements that are tailored for portability. For instance, in one embodiment, the device uses Time Division Multiplexing (TDM), Time Division Switching or Wavelength Division Multiplexing (WDM) to reduce the number of required fiber optic cables.

Regarding the issue of accurate computation of Fourier spectral information, an example embodiment of a diagnostic device includes a Digitization Subsystem that implements an Analog-to-Digital Converter (ADC) having a high number of bits per sample (of the order of sixteen bits per sample) and configured with a sampling rate based on a maximum frequency of a demodulated waveform, e.g., constrained by the Nyquist Rate. In one embodiment, the demodulated waveform is subjected to analog signal processing, e.g., mixed with an additional waveform that centers the resulting waveform at an intermediate frequency, f₁, and the sampling rate is at least f₁+fₘ, where fₘ is a frequency of a modulation waveform. The mixing to the intermediate frequency enables better filtering and amplification. To enhance further the accuracy of the computations, the effects of noise and interference may be compensated for by sending multiple copies of the same modulation waveform over time or by using different angles of incidence.

Regarding the issue of providing meaningful output to a physician, the diagnostic device may analyze multiple sets of optical parameter measurements obtained over time to output an indication of the probability that the patient is responsive to therapy. The analysis can be performed using Artificial Intelligence (AI), e.g., a neural network or an expert system, to output confidence levels as to whether cancerous tissue is detected, and if so detected, whether the therapy is successful. However, the “raw” optical parameters of Absorption and Scattering behind such a presentation can be archived for access by the physician. In some embodiments, the raw information may be displayed in conjunction with the result of the analysis. Archiving the raw information enables the physician to, at a later time, examine the measurements underlying a reported detection and the response to a therapy.

### BRIEF DESCRIPTION OF THE DRAWINGS

FIG. 1 is an example graph illustrating the spectrum of an incident laser beam at a particular frequency.

FIG. 2 is an example graph of the magnitude of the Fourier spectra of received scattered demodulated waveforms from both normal tissue and cancerous tissue as a function of frequency. Gaps are indicated.

FIG. 3 is an example graph of the phase of Fourier spectra of received scattered demodulated waveforms from both normal tissue and cancerous tissue as a function of frequency. Gaps are indicated.

FIG. 4A is an example graph illustrating the spectra of a plurality of incident laser beams at different wavelengths.

FIG. 4B is an example graph illustrating ideally shaped curves of Absorption as a function of wavelength for normal tissue and cancerous tissue when the laser beams of FIG. 4A are applied. Gaps are indicated.

FIG. 4C is an example graph illustrating ideally shaped curves of Scattering as a function of wavelength for normal tissue and cancerous tissue when the laser beams of FIG. 4A are applied. Gaps are indicated.

FIG. 5 is a block diagram of a system for cancer diagnosis according to an example embodiment of the present invention.

FIG. 6A is a block diagram of system components included in an Optical-Electronic Subsystem according to an example embodiment of the present invention.

FIG. 6B is a block diagram of system components included in a Digitization Subsystem and a Parameter Computation Subsystem according to an example embodiment of the present invention.

FIG. 7A is an example graph illustrating non-ideally shaped curves of Absorption as a function of wavelength for normal tissue and cancerous tissue when a plurality of laser beams are hypothetically applied.

FIG. 7B is an example graph illustrating non-ideally shaped curves of Scattering as a function of wavelength for normal tissue and cancerous tissue when a plurality of laser beams are hypothetically applied.

FIG. 8A is an example graph illustrating a result of applying a gap filling technique to the curves of FIG. 7A according to an example embodiment of the present invention.

FIG. 8B is an example graph illustrating a result of applying a gap filling technique to the curves of FIG. 7B according to an example embodiment of the present invention.

FIG. 9 is an example Neural Network.

FIG. 10 is a block diagram of computation elements within a node in an example Neural Network.

FIG. 11 is a block diagram of a Neural Network according to an example embodiment of the present invention.

FIG. 12 is a block diagram of an Expert System according to an example embodiment of the present invention.

FIG. 13 is a flowchart of a method for cancer diagnosis according to an example embodiment of the present invention.

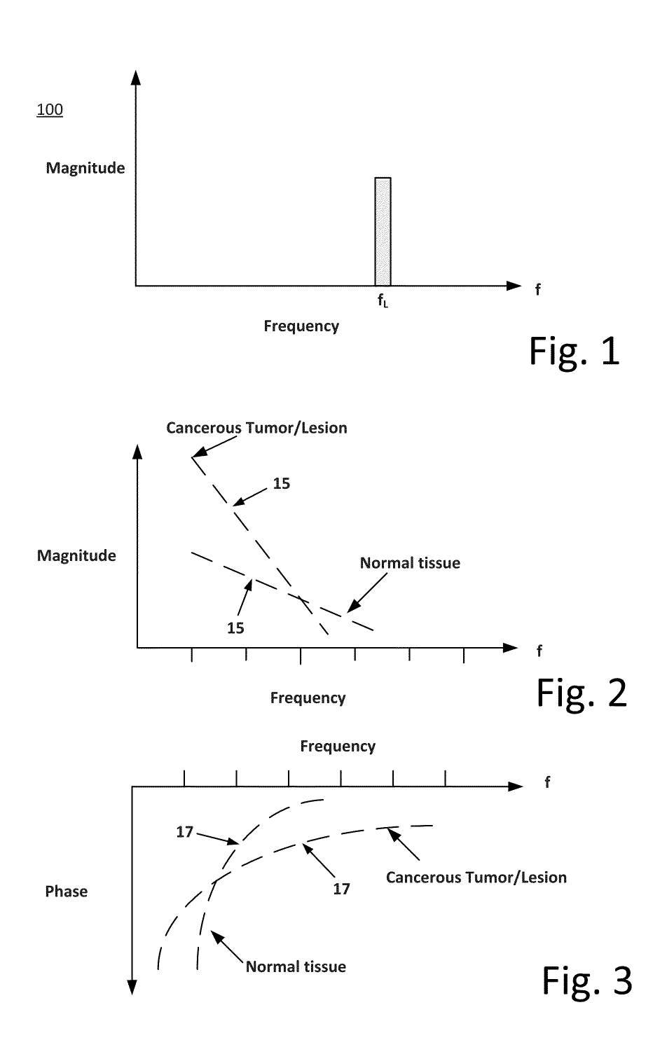

### DETAILED DESCRIPTION

The present invention relates to a portable cancer diagnostic system and device that includes a plurality of subsystems, each of which can be implemented in hardware, software or a combination thereof.

### System Overview

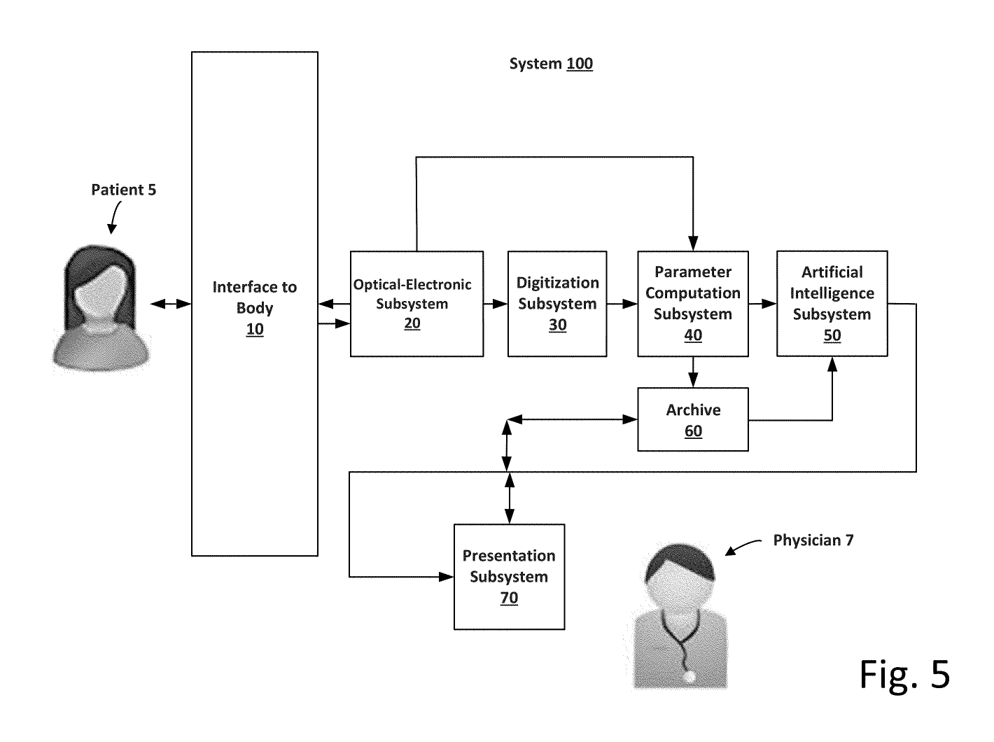

FIG. 5 is a block diagram of a system 100 for cancer diagnosis according to an example embodiment of the present invention. The system 100 may be implemented as a single, portable cancer diagnostic device. Alternatively, the system 100 may be implemented as a plurality of separate components that may be in wired or wireless communication with each other. The system 100 may include an Interface 10 to a patient 5, an Optical-Electronic Subsystem 20, a Digitization Subsystem 30, a Parameter Computation Subsystem 40, an Artificial Intelligence Subsystem 50, an Archive 60 and a Presentation Subsystem 70. Each of these subsystems will be described separately. However, it will be understood that subsystems can be combined into a single hardware and/or software arrangement. Alternatively, components may be remotely located from each over. For example, components may carry out processing via Cloud computing using the Internet.

The Interface 10 may be a wearable or otherwise unobtrusive appliance placed over an area of the patient's body such that the appliance covers and illuminates both tissue which is known to be normal and tissue which is either known or suspected of having a cancerous tumor. The area of the body so covered will be referred to herein as the “active area.” The Interface 10 may include a plurality of fiber optic cables that convey coherent light of different wavelengths, λ to the active area.

The Optical-Electronic Subsystem 20 may include circuitry that generates the coherent light that illuminates the active area. Specifically, this may include hardware that outputs a plurality of laser beams of different center frequencies, i.e., of different wavelengths. Each laser beam may be intensity (amplitude) modulated over time, at a succession of different modulation bandwidths each for a certain time duration, and is coupled into one of the fiber optic cables in the Interface 10. The Optical-Electronic Subsystem 20 may collect and demodulate reflections from the Interface 10 of each modulated laser beam into a fiber optic cable. The Optical-Electronic Subsystem 20 may convert the collected light to an analog electrical format ready for subsequent digital conversion. The Optical-Electronic Subsystem 20 may provide analog information concerning the actual modulated laser beams to the Parameter Computation Subsystem 40 for further use. The illumination can be carried out for both known normal tissue and suspected cancerous tissue in sequence, e.g., illuminating the normal tissue first, then switching to illuminating the suspected tissue. Alternatively, both tissue types can be illuminated simultaneously, e.g., using two laser sources of the same wavelength. Furthermore, in principle, illumination can be carried out for different angles of incidence. The Interface 10 may facilitate this through a mechanism that enables the angle of individual fiber optic cables to be adjusted manually or automatically to a specified angle with respect to a body of the Interface 10. Alternatively, the Interface 10 may itself be repositioned with respect to the body of the patient 5. In one embodiment, the Interface 10 may, by default, be configured so that the fiber optic cables are at different angles.

The Digitization Subsystem 30 receives an analog signal in electrical format from the Optical-Electronic System 20. The analog signal represents a reflected laser beam. The Digitization Subsystem 30 carries out an analog-to-digital conversion on the analog signal. This conversion may be carried out at a sampling rate commensurate with the bandwidth of the analog signal. This conversion may also be carried out with a number of bits per sample commensurate with the relatively high level of accuracy demanded for the computation of Fourier spectral information (Magnitude and Phase) and the corresponding optical parameters of Absorption and Scattering. Preferably, the conversion uses on the order of sixteen bits per sample.

The Parameter Computation Subsystem 40 takes the digitized samples provided by the Digitization Subsystem 30 and computes the Fourier spectral components of the corresponding reflected, demodulated laser beam. These spectral components correspond to magnitude and phase information. The Parameter Computation Subsystem 40 may compute the spectral components using standard Digital Signal Processing (DSP) methods such as a Fast Fourier Transform. The Parameter Computation Subsystem 40 may receive information from the Optical-Electronic Subsystem 20 concerning the corresponding incident modulation waveform. It then computes (in the same manner as for the reflected demodulated laser beam) the corresponding Fourier components, both the magnitude and phase of the incident modulation waveform. Using the results of both of these computations, the Parameter Computation Subsystem 40 computes the ratio of the Fourier components of the collected reflected demodulated laser beam to the incident Fourier components for each modulation waveform. Both magnitude and phase are taken into account in this computation. In doing this it takes into account any frequency shifts required for processing. The result is an estimate of the transfer function relating the reflected demodulated laser beam to the incident modulated laser beam. This transfer function will have its domain, as is typical of Fourier components, in the frequency domain. Henceforth, it will be referred to as the “Fourier Transfer Function.”

The estimate of the Fourier Transfer function is carried out for a particular laser beam (center frequency, “f” or equivalently wavelength, “λ”) over all the modulation frequencies. This produces a collection of Fourier Transfer functions for the particular laser beam. The Parameter Computation Subsystem 40 may then apply the collection as input to an approximation-smoothing process that produces one continuous estimate of the Fourier Transfer function corresponding to the particular laser beam over the entire domain of frequencies presented by all of the modulation waveforms. Carrying out this approximation-smoothing is important. The Fourier Transfer function spectra are the result of the reflectance from human tissue and this reflectance should be smooth. There should be no bumps or discontinuities in its magnitude or in the first derivative of the magnitude with respect to frequency. This approximation-smoothing removes anomalies that may be caused by either measurement or the digital signal processing itself If these anomalies are not removed they would propagate through the subsequent signal processing and may likely affect the ultimate outputs of the system—causing misdiagnoses and inaccurate assessments of therapeutic responses. The smoothed Fourier Transfer function is then used as the input to a computational block which has a diffusion model that converts the Fourier Transfer function into the Absorption and Scattering parameters. One example of a diffusion model is described by Bevilacqua et. al., Broadband absorption spectroscopy in turbid media by combined frequency-domain and steady state methods, Applied Optics, Vol. 39, No. 34, December 2000.

As pointed out earlier, the computed Absorption and Scattering parameters, when considered over a plurality of laser beams, will have gaps in the wavelength domain. These optical parameters may be provided to a computational block within the Parameter Computation Subsystem 40 that uses additional approximation and smoothing techniques to fill in the gaps. Gap filling is extremely useful because it is easier for a physician to understand the optical parameters by looking at continuous curves as opposed to looking at discontinuous curves with gaps. There are no gaps in the actual reflections from human tissue. Instead, there will be a smooth, continuous, monotonic amplitude and continuity of the first derivative with respect to wavelength. If the gaps were allowed to remain in the optical parameters, they could present subtleties which affect the subsequent processing and diagnostic accuracy of the Artificial Intelligence Subsystem 50, discussed next. Accordingly, removing these artifacts would bring the optical parameters in alignment with what is truly expected from human tissue. The Parameter Computation Subsystem 40 therefore outputs estimates of the optical parameters, which are continuous over the full range of wavelengths, and over all lasers. The estimates of the Absorption and the Scattering may be computed for both the normal tissue and the suspected cancerous tissue. Estimates may be computed for different angles of incidence of the same laser beam or for a plurality of laser beams with different angles.

These Absorption and Scattering estimates may be stored in the Archive 60, which may be implemented as a database on any physical storage medium. They are also provided to the Artificial Intelligence Subsystem 50, which may analyze current Absorption and the Scattering estimates in addition to previous estimates from the same patient. The Artificial Intelligence Subsystem 50 may obtain the previous estimates from the Archive 60. The Artificial Intelligence Subsystem 50 may be configured as either a Neural Network or an Expert System, either of which can determine whether or not the suspected cancerous tissue is in fact cancerous and with what probability.

The Artificial Intelligence Subsystem 50 may produce two classes of outputs. First, if the interest is in a basic cancer screening, it may indicate whether 1) The patient is free of cancer and no further action is required, 2) The patient needs further “aggressive” action (e.g., biopsy) or 3) The analysis was inconclusive such that the patient should be tested through additional imaging techniques, e.g. mammography. Second, if the interest is in determining the appropriateness of therapy on previously detected cancer, the Artificial Intelligence Subsystem 50 may determine whether the therapy program is converging to success or not, and with what probability. Neural Networks are trained using previous determinations. The data needed for such training can be stored in the Archive 60. Determinations for current applications to patients can be stored in the Archive 60 and used as an additional source of training data.

Unlike Neural Networks, Expert Systems to do not have to be trained, but rather model the reasoning employed by a human expert—in this case a physician—as a computer algorithm that produces the determination. A great deal of progress has been made in the development of Expert Systems due to increases in the speed and memory of computers, the organization of the hierarchy of databases and the ability of computers to extract knowledge directly from text without much manual intervention. Although the expert's reasoning has been automated, the expert may still play a role in the determination process by, for example, manually identifying appropriate data sources, which can be stored in the Archive 60.

The Presentation Subsystem 70 may provide results concerning cancer detection or success of therapy to the physician and with appropriate confidence measures, using a graphical display interface. If the physician wants to see the sequence of optical parameters, the Absorption and Scattering, which led to the determinations then these optical parameters can be requested from the Archive 60 through the Presentation Subsystem 70. The system may allow the physician to access the presentation on the integrated display, or remotely and wirelessly, for example, by smart phone or tablet computer.

Although presented as a stand-alone device/system, nothing prevents the example embodiments of the present invention from being integrated with systems that use other cancer screening/diagnostic techniques, such as mammography, MRI, ultrasonography, thermography and irradiation by microwaves. The present invention is well adapted to being integrated with other techniques in a physician's or technician's workstation. Such a workstation may well benefit from the economies obtained by integrating different techniques together. In one embodiment, diagnostic determinations made according to the example embodiments could be used to enhance images taken via the other techniques, or even images taken by a camera that takes photographs of the tissue of interest in the human-visible spectrum. For example, the system may overlay images or text onto an image of the actual tissue and include in the overlay internal maps indicating cancerous, non-cancerous and ambiguous areas.

### Optical-Electronic Subsystem

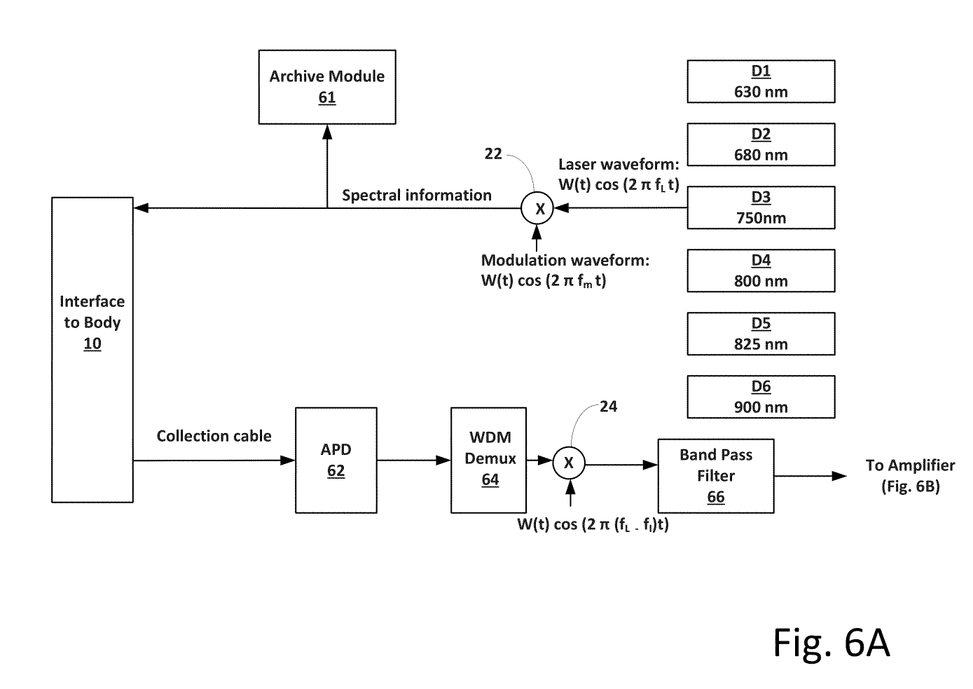

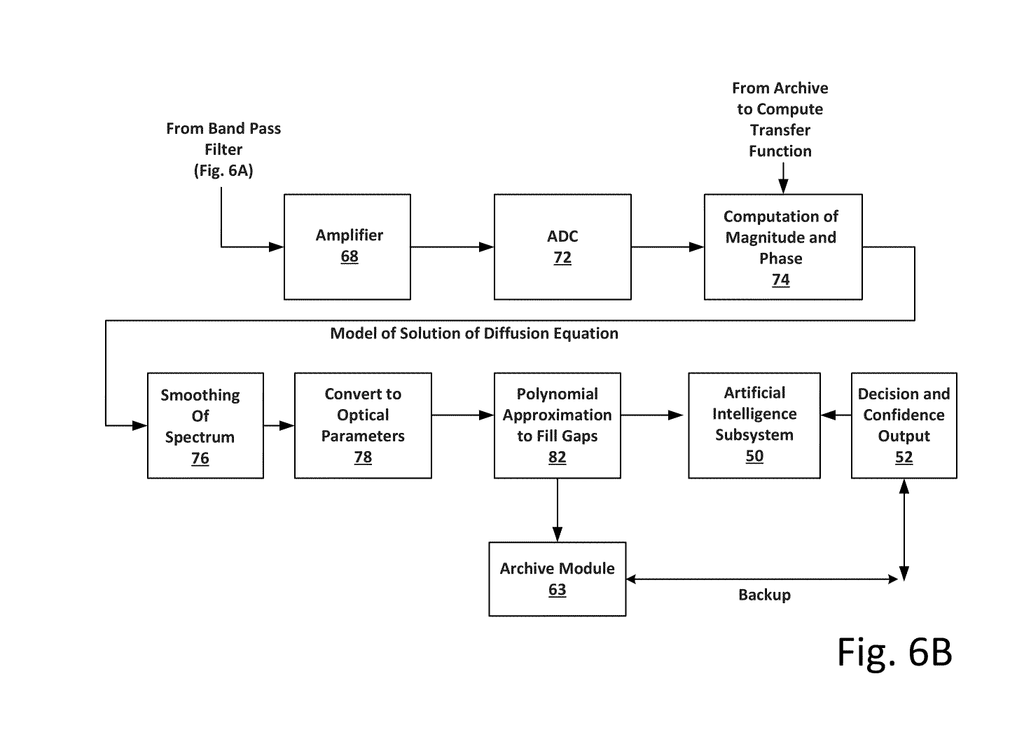

FIG. 6A is a block diagram of system components included in the Optical-Electronic Subsystem 20, which may include a bank of diode lasers D1-D6, each of which emits light at a different wavelength. For example, the wavelengths could be 630 nm, 680 nm, 750 nm, 800 nm, 825 nm and 900 nm. The corresponding carrier frequency associated with each laser is denoted by “f_{L}.” The lasers D1-D6 could be operated sequentially, one laser at a time. Alternatively, they could be operated simultaneously, in which case multiplexing and demultiplexing would be used to separate their transmissions and receptions.

Each of the lasers D1-D6 is intensity, i.e., amplitude, modulated by a sinusoidal waveform of frequency, “fₘ.” The modulation may be performed using a first mixer 22. In the modulation waveform, “W(t)” denotes a time window with a particular duration, “T”. T typically is of the order of a few microseconds, for example, 2 μsec. A given laser emits a continuous sequence of such modulated outputs. Each modulated output corresponds to a different fₘ, where fₘ varies from a floor value of fₘ₁ to a ceiling value of fₘₕ in stepped increments of ΔHz. Specifically, the sequence steps through a range of fₘ values from the floor value of fₘ₁ to the ceiling value of fₘₕ, in step increments of ΔHz. For example, fₘ₁ can be 20 MHz, fₘₕ can be 100 MHz and Δ can be 0.5 MHz, so that the entire transmission from a given laser takes 0.32 msec (assuming no repeated frequencies). For a given laser the time domain signal structure of each modulated laser beam is provided to an Archive Module 61, which may compute the corresponding Fourier spectral information, i.e., magnitude and phase, using DSP techniques such as the Fast Fourier Transform (FFT) and store the results in the Archive 60. It should be noted that while some discussions of DOS have employed values of fₘ as high as 400 MHz, there are diminishing returns for using such high frequencies due to high attenuation of the reflected signal.

Each modulated laser beam is output to the Interface 10 using, for example, a dedicated fiber optic cable for each laser, e.g., six cables in this instance. However, it is also possible to use fewer cables than the number of lasers, in fact a single cable could be shared by all the lasers. To accommodate cable sharing, an example embodiment involves TDM, Time Division Multiplexing or Time Division Switching—in which case each laser obtains the fiber optic cable on a dedicated basis for a given interval of time. This sharing can also be accomplished on a wavelength basis, using WDM, Wavelength Division Multiplexing.

TDM is an effective way to reduce the requirement for a multiplicity of fiber optic cables. However, it increases the time required in the transmission of the radiating signals and the collection of the reflected signals. This does affect the overall throughput in terms of end-to-end processing time. WDM is also effective for reducing the number of fiber optic cables and has no penalty in terms of reduction in throughput due to increased transmission and reception times. Therefore, WDM is preferred for use in connection with the example embodiments. Wavelength Division Multiplexers may be used to combine light of different wavelengths into a single fiber. Multiple wavelengths can be accommodated by WDM, in a manner similar to the frequency division multiplexing that has been used in broadcast radio systems. However, instead of operating on lower frequency radio signals, WDM operates on much higher frequency laser signals. WDM can easily accommodate the combining of the six laser signals in FIG. 6A.

With WDM the incident light from each fiber may be collimated. The collimated beams may then be combined using a dichroic filter before being focused into a target fiber. In the context of the example embodiments, the distances that the optical signals have to traverse is very small, e.g., on the order of a meter, so that amplification prior to the signals being input to a Wavelength Division Multiplexer may be unnecessary. Accordingly, the Wavelength Division Multiplexer can be implemented as a miniature circuit, the dimensions of which are on the order of a centimeter, making it well suited for placement on a printed circuit board. Such miniaturization may be accomplished using various techniques. For example, collimators with plano-convex rod lenses of different radii can be employed. Twisted and parallel fusion processes with fused biconical taper (FBT) techniques may also be employed.

The following example describes the characteristics of a miniature Wavelength Division Multiplexer that could be used in connection with the example embodiments:

Number of wavelengths combined: 6

Operating wavelengths: 630 nm, 680 nm, 750 nm, 800 nm, 825 nm, 900 nm

Fiber Optic cable types connected: multi mode

Fiber core/cladding size (microns): 85/125

Fiber jacket type: Hytrel

Connector type: ST

Back reflection level: 35 dB

As mentioned earlier, the resultant modulated beam from a given laser illuminates both normal tissue and suspected cancerous tissue. Illumination could be simultaneous for both tissue types. It could also be at different times for the different tissue types. Multiple targets for the reflection of each tissue type may be used in the sequence of transmissions, e.g., laser scanning different portions of the active area in a sequential manner. The angle of incidence could also be varied. All of this can be accounted through appropriate processing.

The fiber optic cables may be terminated by solid silicone or immersion in a liquid with known optical characteristics, i.e., a liquid phantom. An example cable diameter is 400 μm.

From a given laser transmission (or simultaneous transmissions if multiplexed) the received scattered light may be collected by a specific fiber optic cable that has a space or gap between it and the tissue facing side of Interface 10. This collection cable could, for example, be a 3 mm solid core cable. The light collected by this cable may then be provided as an input to an Avalanche Photo Diode (APD) 62 suitable for the wavelengths being considered. The APD may be an active-area avalanche photodiode. If WDM has been carried out then a corresponding demultiplexing is performed by a WDM Demultiplexer 64 to separate the laser receptions for processing.

For a given laser transmission modulated with a carrier frequency f_{L}, the received scattered waveform may be brought to an Intermediate Frequency (IF, denoted “f_{I}”) by a second mixer 24. Mixing to f_{I} provides for better filtering and amplification. An example value of the IF frequency is f_{I}=120 MHz. The image of the mixer output around 2 f_{L} is then removed by a Band Pass Filter 66 centered around f_{I} with appropriate cut-off frequencies. This mixing and Band Pass Filter processing essentially demodulates the reflected portion of the Intensity/Amplitude Modulated laser beams.

The resultant demodulated laser beam is provided to an Amplifier 68, shown in FIG. 6B, which amplifies the demodulated signal to put it into an acceptable range for the subsequent analog-to-digital conversion. The amplifier gain may vary depending upon the output power of the lasers. An example output power is 20 mW, which is equivalent to 13 dBm. An example value for the amplifier gain is “10.”

### Digitization Subsystem

FIG. 6B is a block diagram of system components included in the Digitization Subsystem 30 and the Parameter Computation Subsystem 40. The Digitization Subsystem 30 may include an ADC 72 (FIG. 6B) that computes the Fourier spectral characteristics (magnitude and phase) with a high degree of accuracy so that the corresponding optical parameters, Absorption and Scattering are also highly accurate. Practical levels of high accuracy are necessitated due to the fact that accuracy level affects the ultimate reliability of the detection of cancerous tissue and the reliability of any determination of the responsiveness to therapy. The realization of the ADC is thus driven by the need to have as high a number of bits per sample as possible under the constraints of meeting the Nyquist sampling rate on the bandwidth of the demodulated laser waveform. The demodulated laser waveform may be centered at the IF frequency, f_{I}, and have a maximum frequency of f_{I}+fₘ, so that the single sided bandwidth (from center frequency to one end of the bandwidth range) relative to the Nyquist sampling rate could be f_{I}+fₘ +a margin in the form of a guard band. For the example of f_{I}=120 MHz and fₘ=100 MHz this bandwidth can be 220 MHz then with a guard band of 30 MHz the corresponding example sampling rate is 500 Mega Samples Per Second (MSPS). To assure a sufficiently high level of accuracy, the resolution of the ADC is preferably on the order of sixteen bits per sample.

### Parameter Computation Subsystem

The Parameter Computation Subsystem 40 computes the magnitude and phase of each of the modulated laser beams, for each modulation frequency or modulation wavelength. It was mentioned earlier that this could be carried out in the Archive Module 61. Alternatively, Archive Module 61 could supply the time domain forms of these waveforms to a Computation of Magnitude and Phase module 74 shown in FIG. 6B, where the Fourier Spectral components are computed. These particular Fourier spectral components will be referred to henceforth as “the reference.” The reference is processed so that the Fourier spectral components corresponding to the entire range of modulation frequencies (from fₘ₁ to fₘₕ in steps of ΔHz) are considered and parsed together. A smoothing approximation algorithm 76 is then employed using a polynomial estimation to create a continuous representation of the reference. The resulting estimate is continuous over the entire frequency range and may also have a continuous first derivative so as to better correspond to the actual reflected signals.

Concurrent with (e.g., simultaneously or immediately after) the polynomial approximation, the Computation of Magnitude and Phase module 74 may receive digitized samples of the corresponding waveforms from the Archive 60. These digitized samples are referred to henceforth as the “measured signal” and need not be processed in real time. Instead, they can be put in a mass storage device, a hard disk for example, and then processed upon subsequent command. As mentioned, DSP techniques such as the FFT may be used to compute the magnitude and phase of the corresponding reflected and demodulated laser beam. In like manner to the processing of the reference, these Fourier spectral components may also be parsed together. Likewise, a smoothing approximation algorithm based on polynomial estimation may be used to provide a continuous representation of the spectral components in the measured signal. Such processing is carried out in the “Smoothing of Spectrum” module 76. By way of example, the FFT could be carried out over 2048 samples and performed on processor of an external computer by commercially available software such as MatLab or by a dedicated processing unit in the diagnostic device.

For a given laser beam, once the above processing of the reference and the measured signal has been completed the smoothed estimates of the spectral components of the measured signal may be divided by corresponding spectral components of the reference to form a set of ratios that collectively define an estimated Fourier Transfer Function over the entire range of modulation frequencies fₘ₁ to fₘₕ,. This is carried out using the arithmetic of complex numbers and results in the Fourier Transfer Function, both its magnitude and phase. In this manner, the computations can be carried out for all of the laser beams over the entire range of wavelengths. The transfer function calculation may account for the IF frequency, f_{I} so that the magnitude values are frequency aligned to the phase values.

The output is then provided to a “Convert to Optical Parameters” module 78. The module 78 may use an algorithm based upon a diffusion model to convert the Fourier Transfer function to the optical parameters, Absorption and Scattering. As with the computation of the magnitude and phase values of the Fourier Transfer Function, the optical parameters may be computed by a processor of an external computer or a dedicated processor in the diagnostic device.

To fill in the gaps, the optical parameters may be processed by a “Polynomial Approximation to Fill Gaps” module 82, which uses a smoothing and polynomial fit algorithm to provide a complete and continuous estimate of the Absorption and Scattering over the full wavelength range, for both the normal tissue which was illuminated and the suspected cancerous tissue. This smoothing and polynomial fit assures that there is total continuity in the Absorption and Scattering values and, preferably, also continuity of the first derivative of the Absorption and Scattering values. The results may be output to the Artificial Intelligence Subsystem 50 which, as mentioned earlier, can be a Neural Network or an Expert System. The results may also be stored in the Archive 60 through a second Archive module 63. The Artificial Intelligence Subsystem 50 outputs a decision 52, possibly together with a confidence level. The decision 52 and confidence level may be stored, e.g., in the Archive 60, and fed back to the Artificial Intelligence Subsystem 50 for use in future decision making

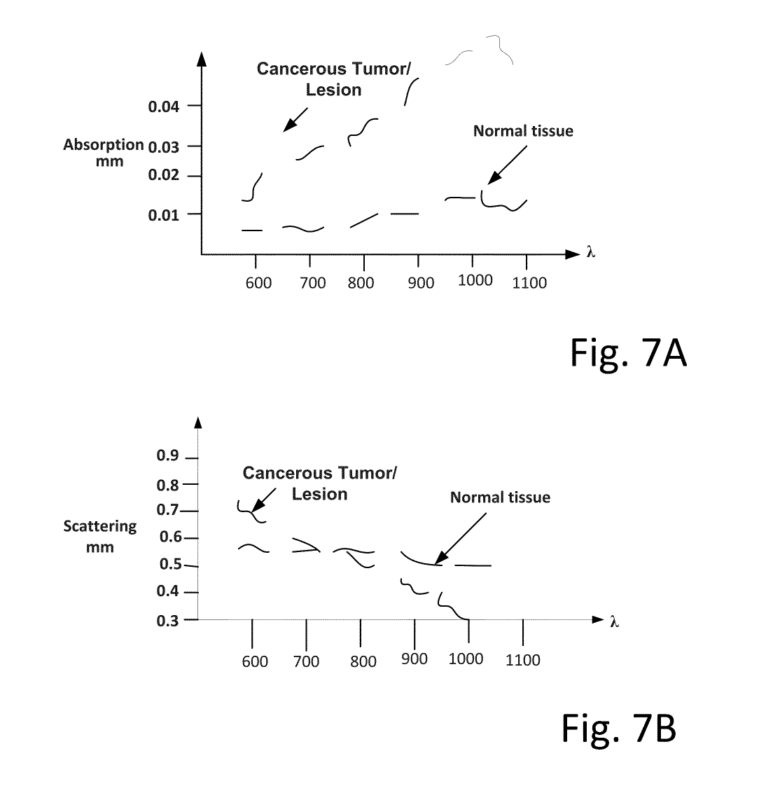

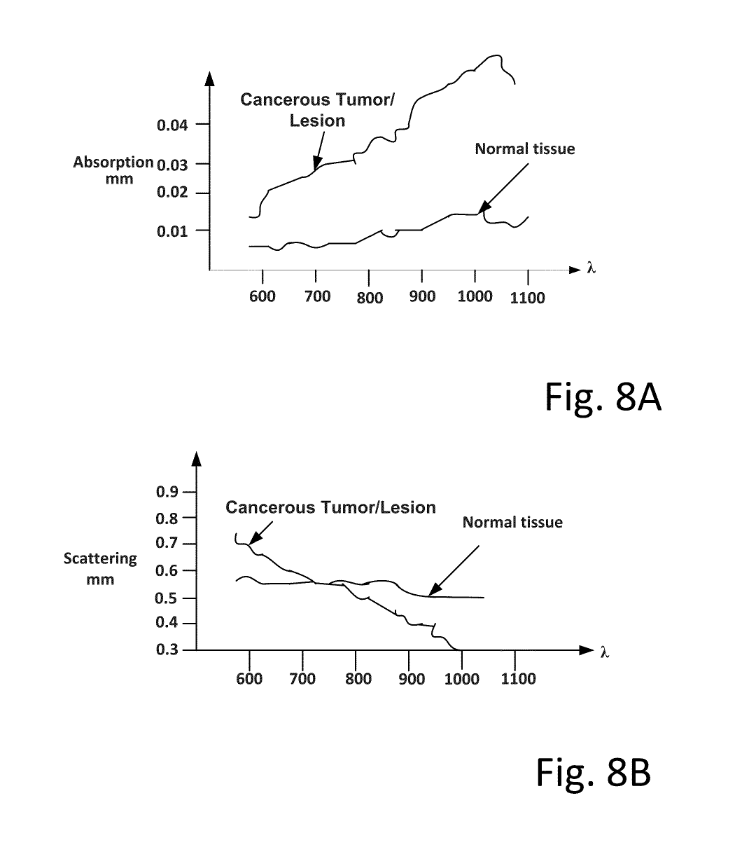

FIGS. 7A and 7B illustrate the Absorption and Scattering as computed with the gaps prior to the operation of the module 82. FIGS. 8A and 8B illustrate the Absorption and Scattering as computed after the module 82 has filled the gaps.

The module 82 may employ at least one of a number of different gap filling techniques. The first technique is as follows. Consider the Absorption data resulting from the six laser beams shown in FIG. 7A, which may correspond to the lasers D1-D6. The corresponding continuous segments of the Absorption data resulting from each laser are represented respectively by A1(λ), A2(λ), . . . A6(λ); where each Ai(λ) is a continuous function of λ. Each Ai(λ) has bounded support and the different Ai(λ) not only do not overlap, but there are gaps between their supports, as noted. For simplicity, assume that each of the supports is “Δλ” wide in wavelength units, e.g., 100 nm. Furthermore, assume that each Ai(λ) is sampled every [Δλ]/N. For example, N could equal 100 in which case there would be 100 samples of each Ai(λ). These can be denoted as follows:

A11, A12 . . . A1N - - - corresponding to Laser D1 and to samples taken at λ11, λ12, . . . λ1N,

A21, A22 . . . A2N - - - corresponding to Laser D2 and to samples taken at λ21, λ22, . . . λ2N,

A61, AN2 . . . A6N - - - corresponding to Laser D6 and to samples taken at λ61, λ62, . . . λ6N.

Consequently, there are six groups of samples and 6N samples altogether. A polynomial of a specific degree, “m₁” can be determined which will approximate this entire group of 6N samples to within a certain degree of error. The degree of error will decrease as m₁ increases. The type of polynomial may be chosen so as to minimize a particular measure of error. For example, Chebyshev polynomials may be used to minimize the maximum error and Bernstein polynomials for another measure of error.

Lagrange Interpolation polynomials may be used as an alternative. This class of polynomials has the property that the error is exactly equal to zero at the sample points above—though not necessarily at other points. To see how these are generated, consider the “λ” sampling points above, which for discussion purposes are relabeled as: λ1, λ2, . . . λ6N. The corresponding Absorption samples are relabeled as AB1, AB2, . . . AB6N. The Lagrange Interpolation polynomial, p(x), of degree “n” is then given by the following formula:

p(λ)=Σ{Π[(λ−λj)/(λi−λj)]}ABi

Here the sum extends from i=1 to n+2. The product extends from j=1 and j≈i to j=n+2.

A second gap filling technique is a generalization of the first technique above, and has sometimes been referred as “Non-Uniform Sampling” or “Compression” sampling. It basically forms an approximation using the Lagrange Interpolation polynomials. However, it does this by first sampling the Absorption and Scattering at values of λ where there are no gaps. The sampling occurs at a high rate (at least twice the Nyquist rate).

A third gap filling technique uses an approximating polynomial known as a “Spline Function,” which is a piecewise polynomial of degree “m₂” formed by connecting polynomials together at points referred to as “knots.” These have “m₂−1” continuous derivatives. The piecewise nature allows better approximation when the physical parameters change significantly from region to region. This may well be the case for the physical parameters being considered here, i.e., the optical parameters of Absorption and Scattering.

Other gap filling techniques which may be applied in connection with the example embodiments do not use polynomial approximation.

A first non-polynomial technique is based upon the fact that the Absorption and Scattering parameters are generally well-behaved, continuous and with continuous derivatives dispersed throughout the wavelength range. Therefore the technique of “analytic continuation” can be applied. This is a property of complex functions where if the value of the function is known on a finite set of points then it can be determined everywhere. In other words, in this case it is known where there are no gaps and as a result it can be extended through the gaps. This is usually done by employing infinite series to determine the function.

A second non-polynomial technique is based upon what is known as “Maximal Entropy Spectral Estimation.” This fills in the gaps in a way that the resulting Fourier Transform relative to a parameter “θ” is as random as possible.

A third non-polynomial technique is based upon the Whittaker—Shannon interpolation formula, also known as sinc interpolation. This is a method generally used to construct a continuous-time band-limited function from a sequence of real numbers. It can be applied here as well.

Any one of the above techniques can be used for gap filling. Multiple techniques may also be used in combination. If multiple techniques are used, the resulting gap-filled approximations of the Absorption and Scattering parameters produced by each technique could be separately supplied to the Artificial Intelligence Subsystem 50 for analysis.

### Artificial Intelligence Subsystem

The Artificial Intelligence Subsystem 50 may be implemented as a Neural Network or an Expert System. For purposes of simplifying the description, it will be assumed that only one polynomial approximation technique is employed for both smoothing the Absorption and Scattering parameters and filling the gaps. However, the following discussion of the Artificial Intelligence Subsystem 50 is readily generalizable to situations involving multiple approximation techniques.

A Neural Network is a computing device that processes input using artificial intelligence to produce outputs. However, it is not programmed in the conventional sense, i.e., via explicit program instructions usually referred to as the stored program or Von Neumann architecture. Rather, it is trained by giving it examples of inputs and corresponding outputs. A typical training set may contain many examples. As a result the network adapts its internal logical structure so that when it is provided with a non-example input it will produce a corresponding output based on its prior training.

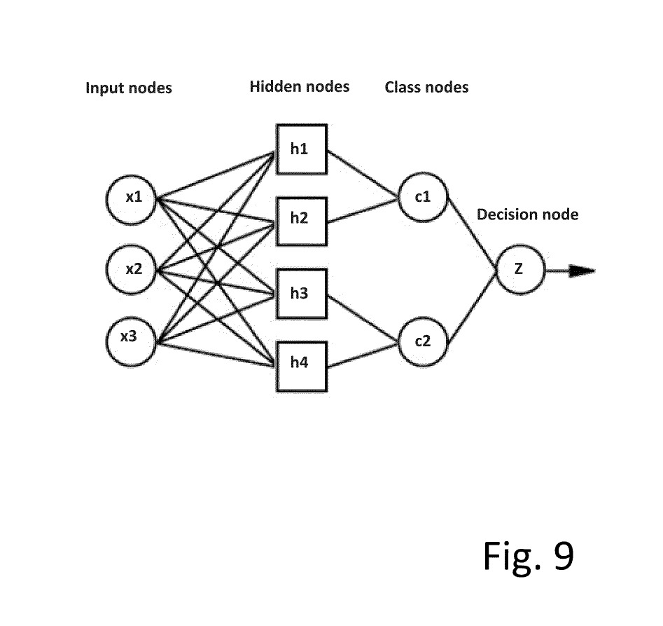

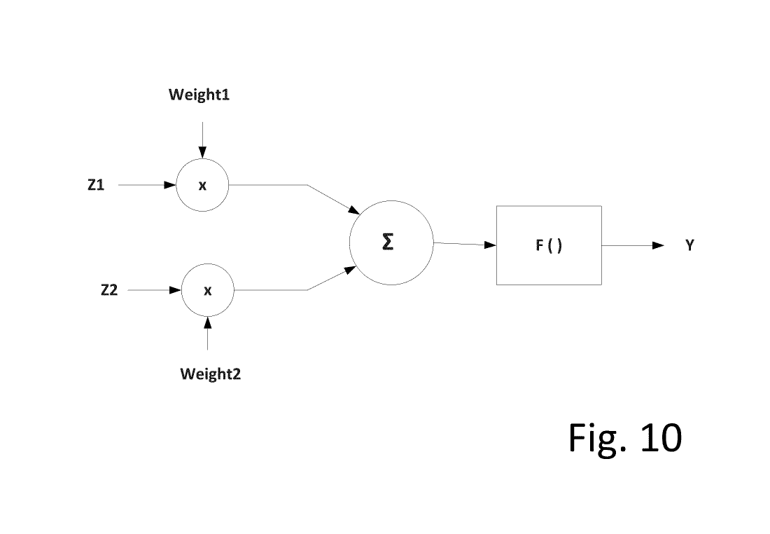

There are many structures and topologies of Neural Networks. FIG. 9 shows one example in which inputs are provided to Input Nodes, x1-x3, and an output is generated by a single Decision node, Z. A layer of Hidden Nodes, h1-h4, connects the input nodes to another set of interior nodes called Class nodes c1-c2, and these then connect to the single output node, the Decision node, Z. The two layers of hidden nodes and Class nodes are, in fact, all “hidden nodes.” Computation occurs in each node. The structure of the node and the nature of this computation are illustrated by way of example in FIG. 10, where Z1 and Z2 are shown as inputs to respective nodes. Z1 and Z2 are each multiplied by a corresponding weight, Weight1 and Weight2. The resulting products are then added together. The resulting sum is then applied to a function block, F( ) that produces the output, Y as a function of the sum. The function F( ) may be linear, e.g., a multiple of the sum. Alternatively, F( ) could be the sign of the sum, Sgn( ) or some other non-linear function. The values of the weights in all of the nodes in a Neural Network are determined through the training previously mentioned. Depending upon the particular application, there may be multiple layers of hidden nodes and multiple outputs with different functions F( ).

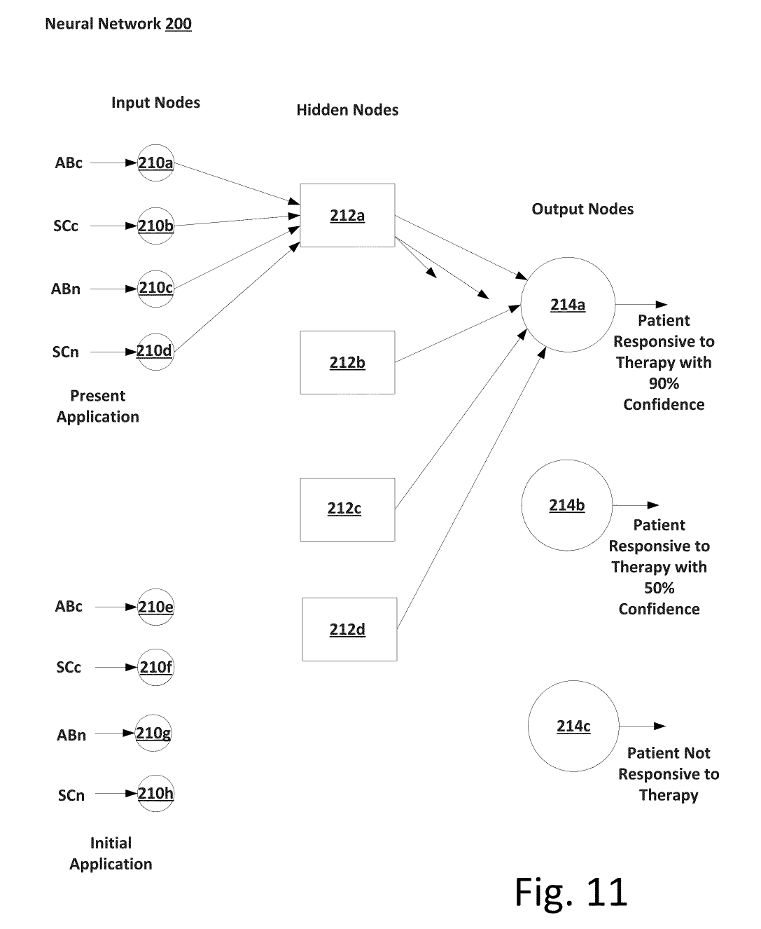

FIG. 11 shows a Neural Network 200 according to an example embodiment of the present invention. The Network 200 includes three layers. On the input side, there are groups of input nodes 210. Each group includes four input nodes 210 and corresponds to a single application of the diagnostic device to the patient. For simplicity, only an initial application and a present application are shown. Each of the input nodes 210 in a group corresponds to the optical parameters estimated, that is:

ABc: Absorption, suspected cancerous tissue

SCc: Scattering, suspected cancerous tissue

ABn: Absorption, normal tissue

SCn: Scattering, normal tissue

In practice, each of the individual input nodes 210 may comprise a plurality of nodes corresponding to a plurality of samples of the Absorption and Scattering parameters over the entire wavelength range. The samples may include Absorption and Scattering parameters taken for different angles of incidence of the laser beam, over different targets of the tissue and for different repetitions of the same modulated laser beam during the same application. For simplicity, these additional nodes are not shown and are instead subsumed into the input nodes 210.

[86] The following example applies to a single angle of incidence. Assume that N=10 samples are taken of each of the above input optical parameters and that these are denoted as:

ABc1, ABc2, . . . ABc10

SCc1, SCc2, . . . SCc10

ABn1, ABn2, . . . ABn10

SCn1, SCn2, . . . SCn10

There are consequently 40 inputs to the Neural Network in this example. The number of inputs would scale with the number of angles of incidence used for illumination. There is full connectivity in the Neural Network moving from left to right, that is, all nodes are connected, either directly or indirectly, to all nodes on the adjacent layer.

The hidden nodes 212 are analogous to the nodes h1-h4 and c1-c2 in FIG. 9 and implement the hidden logic of the neural network 200.

The output nodes 214 compute confidence levels. By way of example, the outputs may represent three different levels of responsiveness to a therapy. Thus, one of the nodes 214a may be activated to output a decision representing a 90% confidence level that the patient was responsive, another node 214b may represent a 50% confidence level, and yet another node 214c may represent that the patient was not responsive. The outputs may equally have represented three decisions on the detection of cancer, each with a different confidence level.

The Neural Network 200 may operate in one of two modes: a General Mode and an Override Mode. In the General Mode there is data available to train the Neural Network. Training data may be stored in the Archive 60. In the initial application there are only inputs for the nodes representing the present application. All other inputs are zero or null because they do not exist at this time. For subsequent applications, these additional inputs, including the inputs from the initial application, can be made available. Outputs of the neural network may be sent to the Archive 60 for storage, e.g., using the Archive module 63 in FIG. 6B. The stored outputs may be used together with the optical information also as training data, in other words to enhance or extend the existing training data.

In the Override Mode there is no training data available. This may occur in practice for any number of reasons. In the Override Mode, the Neural Network 200 may include weights and F( )) configured so that a simple Euclidean metric is computed between the samples of the normal tissue and the cancerous tissue, e.g. |ABc−ABn| and |SCc−SCn|. Here, the vertical bars | | denote the positive square root of the sum of the squares of the sample differences. These metrics may be presented to the physician by the Presentation subsystem 70 to enable the physician to determine the response to the therapy with an appropriate confidence level. The metrics may also be sent to the Archive 60 for storage and subsequent use as training data in combination with the optical information.

If the physician is only interested in screening the patient to determine if a suspected cancerous tissue requires further examination, e.g., by biopsy or some other technique, then the Neural Network can be appropriately simplified by removing the nodes associated with assessing patient response to therapy.

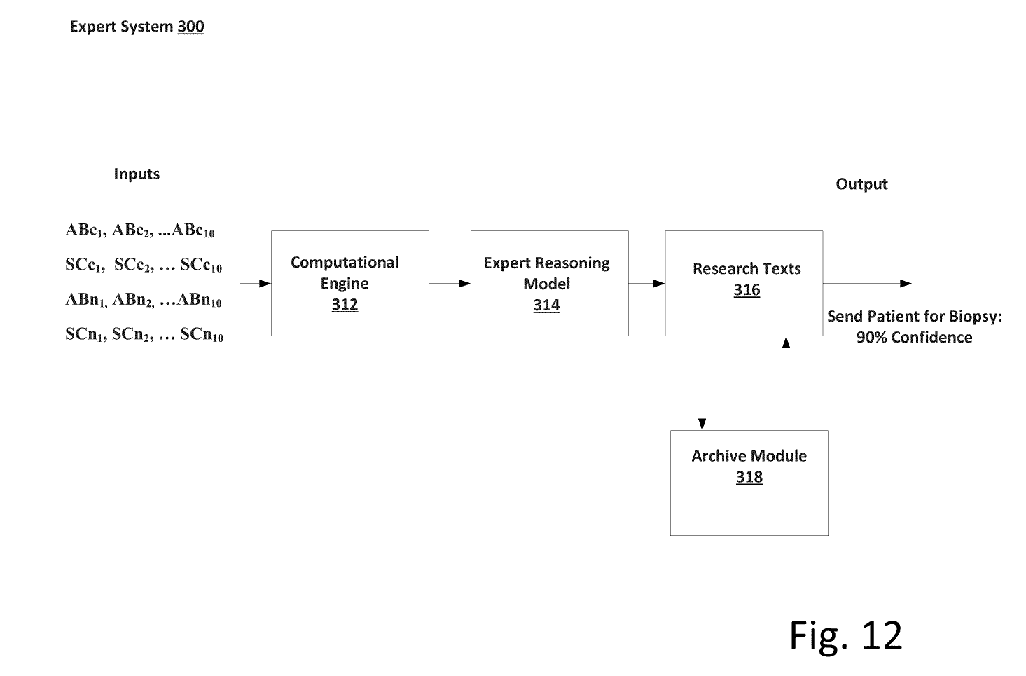

FIG. 12 shows an Expert System 300 according to an example embodiment of the present invention. The Expert System 300 may share inputs in common with the Neural Network 200, specifically: ABc, SCc, ABn and SCn discussed above. As with the Neural Network 200, there may in actuality be a plurality of samples of the Absorption and Scattering parameters over the entire wavelength range, possibly for different angles of incidence, over different tissue targets and for different repetitions of the same modulated laser beam during the same application.

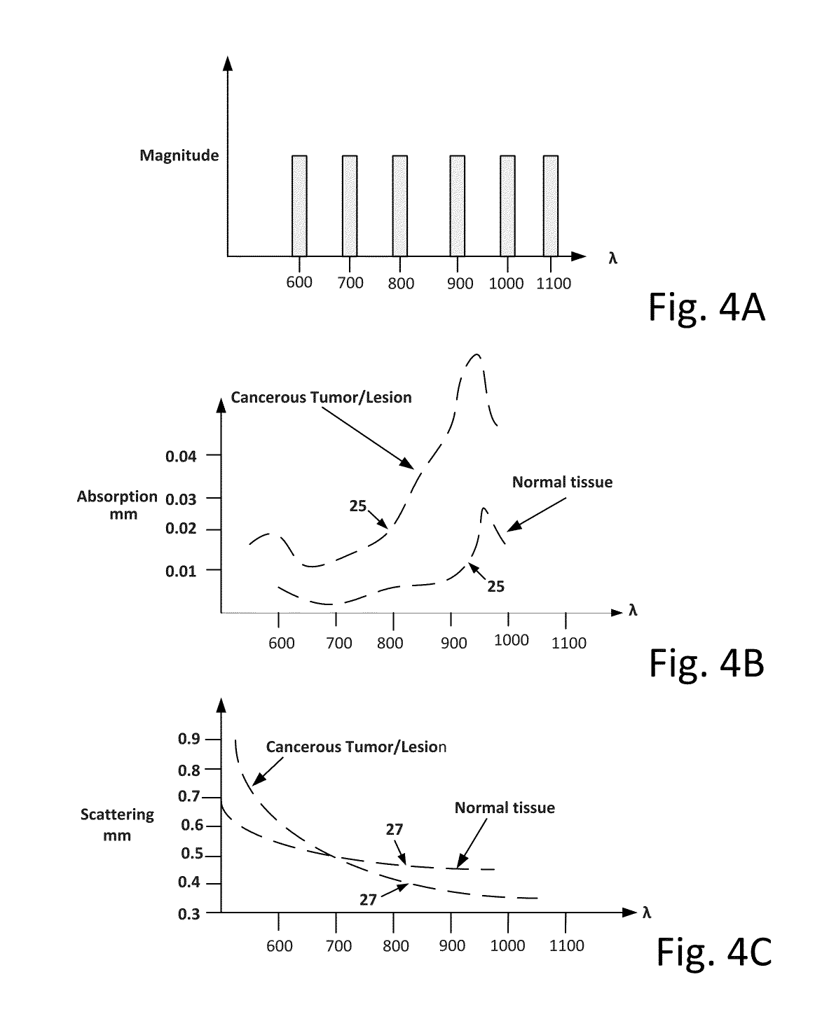

The Expert System 300 may receive as input a pair of curves of the type shown in FIGS. 4B and 4C, and possibly multiple pairs of curves for different incidence angles. The Expert System may include a stored computer program 314 that models the logical decision process of a physician when presented with input data of this type. The Expert System may have access to texts and documents, which are published by recognized experts and relevant to analyzing the optical parameters in the curves. These texts and other documents may be stored in the Archive 60 via the Archive module 318. The Expert System may take the input data and process it through the program 314, which applies the texts and documents. The processing may produce one of three possible outputs.

The following example illustrates how the Expert System 300 may operate. Assume that data has been collected from a single angle of incidence and, as with the above discussion of the Neural Network 200, that there are ten samples of each of the optical parameters:

ABc1, ABc2, . . . ABc10

SCc1, SCc2, . . . SCc10

ABn1, ABn2, . . . ABn10

SCn1, SCn2, . . . SCn10

This is shown as the input to the Expert System on the left of FIG. 12. By way of example, these could be derived from the curves in FIGS. 4B and 4C. The input is sent to a Computational Engine 312, which may compute the differences between successive samples of the corresponding optical parameters of the suspected cancerous tissue and the normal tissue. Specifically, it may compute:

εAB1=ABc1−ABn1, . . . εAB10=ABc10−ABn10

εSC1=SCc1−SCn1, . . . εSC10=SCc10−SCn10

Then for each of these sequences of differences, ε, it may compute an arithmetic average over all ten samples to produce two averages, εAB and εSC, where the underscore symbol “_” represents arithmetic average. The two arithmetic averages may then be provided to the Expert Reasoning model 314, which may implement the following logic:

εAB>0.03 and εSC≦0.02: Send Patient for a biopsy

|εAB|≦0.005 and |εSC|≦0.005: Patient normal—no further action

All other combinations of εAB>0.03 and εSC≦0.02: Send Patient for additional Imaging, e.g., Mammogram

Here, the vertical bars | | symbolize absolute value.

Applying the reasoning above to the data in FIG. 4B or 4C could well result in the first possible output, “Send Patient for a biopsy.” However, because the Scattering data for the normal tissue crosses that of the suspected cancerous tissue in FIG. 4C, this could be a “close choice,” where the modeled reasoning selects one of the possible decisions in favor of the other possible decisions by a small margin.

To enhance the decision making of the Expert System 300, the result of applying the modeled reasoning to the input data may be provided to a Research Texts module 316, which obtains relevant texts/documents from the Archive 60, via the Archive module 318. With the aid of information from the texts/documents, the module 316 may compute a confidence level for the result output by the model 314. The confidence level may be a percentage, e.g., “Send Patient for Biopsy: 90% Confidence,” as shown in FIG. 12.

There are a number of different computer languages that can be applied to implement the programming in the Expert System 300. Examples include the OWL Web Ontology Language, CycL and SNOMED CT. Algorithms written in these languages can be called from inside programs written in more conventional languages like JAVA.

Independent of whether the Artificial Intelligence Subsystem 50 is implemented as a Neural Network or an Expert System, the detailed computations performed by the Subsystem 50 can be carried out in two ways. They may be carried out locally, e.g., utilizing both software implemented algorithms and computational hardware which physically resides at the place where the physician is present. Ideally, most if not all of the hardware and software is co-located in a single diagnostic device. However, the computations can also be carried out externally, by sending the computational inputs to remote software and hardware resources, e.g., to a Cloud computer via the Internet.

The Expert System approach is particularly suited for external computation because Cloud computing resources can potentially access any number of databases containing medical or scientific literature, which will then be used in carrying out the decision making of the Expert System 300. Expert Systems are by their nature very computation intensive and operate on vast quantities of data. Cloud computing is naturally suited to handling large amounts of data and allows the Expert System to separate the various tasks for parallel execution by different computers within the Cloud. For example, consider the reading by computer of textual material relevant to the classification of tissue as either “normal” or “cancerous.” This task may be partitioned into smaller subtasks, for example, a first subtask devoted to computerized reading of material related to genetic factors, a second subtask devoted to computerized reading of material related to environmental factors, a third subtask devoted to computerized reading of material related to immediate medical history, and a fourth subtask of determining whether absorption and scattering inputs similar to those presently being processed have been processed and recorded by the system in the past. These subtasks can all be performed in parallel by Cloud computers to speed up the overall processing.

If Cloud computing is used, the Expert System 300 could coordinate these computers so that each computer first performs a quick, non-exhaustive reading of the material corresponding to the respective subtasks and reports back the findings. The Expert System 300 could examine the initial report and may then continue this reading-reporting process iteratively by issuing commands for continued reading by only those computers whose reports indicated a large quantity of relevant material (e.g., a threshold amount of relevant text or a threshold proportion of relevant text in relation to the amount of text read). Thus, in the language of Graph Theory, the Expert System 300 could “prune” subtasks attached to a common node, thereby purging those subtasks that do not yield relevant material and keeping those that do. The Expert System may then parse together the results of many reading subtasks along with additional sources of relevant information. For example, it may combine the results of the reading subtasks with optical information (Magnitude, Phase, Absorption or Scattering) stored in Cloud computers from past examinations of the patient 5 or optical information for other patients, along with subsequent decisions as to whether the corresponding tissue was shown to be normal or cancerous. Cloud based parallel computing may enable many combinations of data sources to be considered in generating the output of the Expert System 300. Furthermore, the Expert System 300 may be configured to learn from parallel computations so as to optimize its own algorithms to become more efficient, potentially reducing a need for reliance on external data sources in future decision making. Thus, Cloud based parallel computing may facilitate an embodiment where the Artificial Intelligence Subsystem 50 includes features of both a Neural Network and an Expert System, whereby decision making by the Expert Reasoning model is adapted over time, using relevant texts and/or results derived from relevant texts as training data.

### Presentation Subsystem

The Presentation Subsystem 70 may display, on an integrated graphical user interface, the results of the decision making by the Artificial Intelligence Subsystem 50 to the physician. Upon request from the physician, the Presentation Subsystem 70 may obtain from the Archive 60 the entire history of smoothed Absorption and Scattering estimates upon which the Artificial Intelligence Subsystem 50 outputs are based. Upon request, the Presentation Subsystem 70 can also obtain and display the earlier estimates that contain gaps. The graphical user interface may enable remote viewing of the displayed information. For example, the Presentation Subsystem 70 can output the graphical user interface via the Internet or a local network for display at a remote computer such as the physician's personal computer, smart phone, or tablet computer.

As one aspect of the present invention relates to a diagnostic device that can be used by an office-based physician with little additional training, the Presentation Subsystem 70 may format the displayed information in an unambiguous and easily interpreted manner. For example, the display may include an image of the area of the body being tested, together with processed optical information (e.g., the estimates of Absorption/Scattering before or after gap filling), the results of the decision making by the Artificial Intelligence Subsystem 50, and a statistical assessment of the tissue of interest (e.g., confidence level). In particular, a display that distinguishes suspected or confirmed cancerous tissue from normal tissue (e.g., via color coding or another display format) and includes decisions by the Artificial Intelligence Subsystem 50 regarding diagnosis and/or the response to therapy would make interpretation by a human viewer significantly easier compared to displaying the normal and cancerous tissues separately or displaying the tissues without a visual reference to the decisions of the Artificial Intelligence Subsystem 50. If the optical information is included, it can be displayed in a numerical format that is easily interpretable by a physician with no engineering or imaging background.

### Averaging Against Noise and Interference

The sampled spectral information may be susceptible to noise and interference, which prevents reliable detection of cancerous tissue or reliable determination of the success of therapy. Such noise and interference may result from the presence of active components such as the mixers 22 and 24, the Amplifier 68 and the ADC 72. They may include the quantization noise of the ADC as well as noise generated by any non-linearities or other deleterious mechanisms in processing. The effects of external noise and interference can be averaged out by, for example, sending multiple copies of the same modulation waveform but spaced apart in time (time diversity) or using different angles of incidence for the light illuminations (space diversity). The resulting Absorption and Scattering parameters can then be averaged over a period of time to produce approximations that are substantially noiseless and interference free. The effects of the noise related to the quantization may be ameliorated by using non-uniform quantization techniques along with such techniques as “condensed sampling” and “dithering.”

### Method Overview

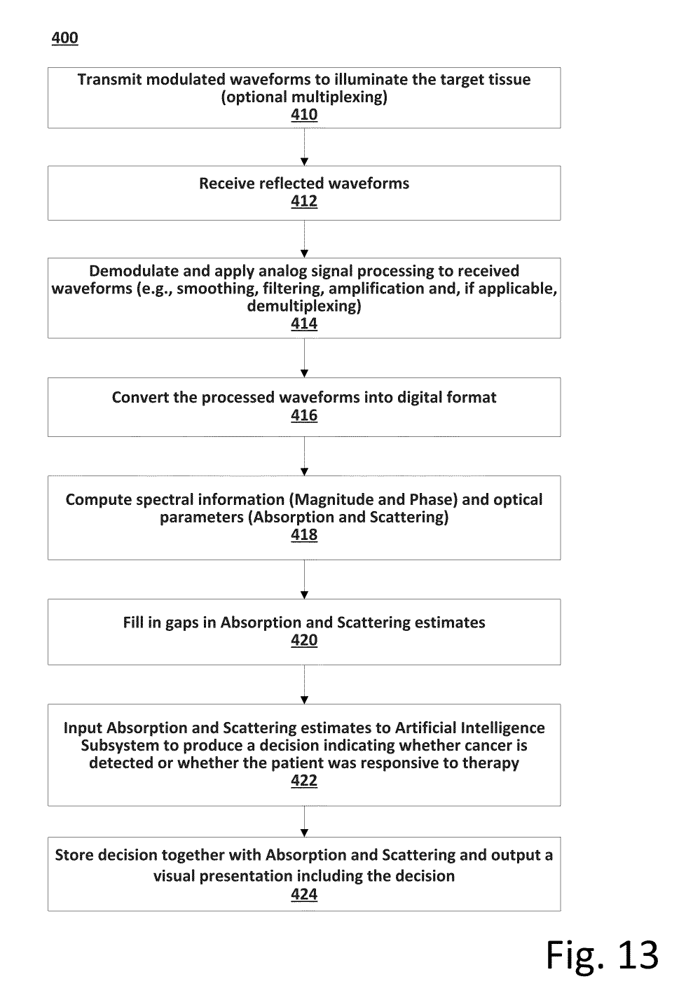

FIG. 13 is a flowchart of a method 400 for cancer diagnosis according to an example embodiment of the present invention. The method 400 may be performed using the system 100. At step 410, the Optical-Electronic Subsystem 20 generates a plurality of amplitude modulated laser beams and transmits those beams through at least one fiber optic cable. The Optical-Electronic Subsystem 20 may transmit the beams using a form of multiplexing such as TDM or WDM, so that the number of cables is potentially fewer than the number of beams. The beams are output at the Interface 10 to illuminate a target area of tissue, which includes at least suspected cancerous tissue or confirmed cancerous tissue. Both normal tissue and suspected/confirmed cancerous tissue may be illuminated simultaneously or in sequence so that the reflected beams capture information for both tissue types, thereby enabling the spectral information corresponding to the normal tissue to be used as a reference with which to compare the suspected/confirmed cancerous tissue. In an alternative embodiment, data for the normal and suspected/confirmed cancerous tissues might be collected through separate applications of the system to the same patient, e.g., by positioning the Interface 10 over normal tissue, then repositioning over an area that includes suspected/confirmed cancerous tissue.

At step 412, the reflected waveforms produced by the reflection of the laser beams off the target tissue, e.g., the active area discussed earlier, are received through at least one fiber optic cable that passes the reflected waveforms to the Optical-Electronic Subsystem 20.

At step 414, the Optical-Electronic Subsystem 20 demodulates the reflected waveforms to obtain analog information, to which it applies analog signal processing, which may include, but is not limited to, smoothing, filtering, amplification and demultiplexing.

At step 416, the Digitization Subsystem 30 performs an analog-to-digital conversion on the analog information processed earlier in step 414.

At step 418, the Parameter Computation Subsystem 40 computes the spectral information (Magnitude and Phase), performs appropriate smoothing and then uses the magnitude and phase information to compute Absorption and Scattering, e.g., by computing a Fourier Transfer Function and converting the Fourier Transfer Function into an estimate of the Absorption and Scattering.

At step 420 the Parameter Computation Subsystem 40 performs a gap filling technique to fill in the gaps in the Absorption and Scattering estimates. As described earlier, the gap filling may involve a form of polynomial approximation.

At step 422, the Artificial Intelligence Subsystem 50 receives the Absorption and Scattering estimates as input. The Artificial Intelligence Subsystem 50 may also obtain additional input in the form of Absorption and Scattering estimates from a previous application of the diagnostic device or system. The input is processed, e.g., using a neural network or expert system, to produce a decision indicating whether cancer is detected or whether the patient was responsive to therapy. The decision may include a confidence level indicating a degree of certainty assigned by the Artificial Intelligence Subsystem 50 to the decision.

At step 424, the decision and the Absorption and Scattering estimates may be stored in the Archive 60 for subsequent use. At this time, the Presentation Subsystem 70 may display the decision and the Absorption and Scattering estimates to a human user, e.g., the physician. If the Magnitude and Phase have also been stored, these may be displayed as well.

An example embodiment of the present invention is directed to one or more processors, which may be implemented using any conventional processing circuit and device or combination thereof, e.g., Central Processing Unit(s), Microprocessors, Field Programmable Gate Arrays (FPGAs) and other signal processing devices, to execute instructions provided, e.g., on a non-transitory, hardware-implemented computer-readable storage medium including any conventional memory device, to perform any of the methods described herein, alone or in combination.

In the preceding specification, the present invention has been described with reference to specific example embodiments thereof. It will, however, be evident that various modifications and changes may be made thereunto without departing from the broader spirit and scope of the present invention as set forth in the claims that follow.

The embodiments described herein may be combined with each other in various combinations. The specification and drawings are accordingly to be regarded in an illustrative rather than restrictive sense.

What is claimed is:

1. A computer-implemented method for cancer detection using Diffuse Optical Spectroscopy, comprising:
illuminating a target area containing suspected or confirmed cancerous tissue, using a plurality of light signals of different wavelength;
at a processor of a computer, processing corresponding reflected signals from the target area to compute estimates of absorption and scattering; and
applying an artificial intelligence system to the absorption and scattering estimates to output a decision as to whether cancer is detected in the target area.

2. The method of claim 1, wherein the artificial intelligence system includes at least one of a neural network and an expert system.

3. The method of claim 1, further comprising:
transmitting the light signals to the target area through at least one transmission cable and using time division multiplexing, time division switching or wavelength division multiplexing, wherein the number of transmission cables is less than the number of light signals.

4. The method of claim 1, further comprising:
amplitude modulating the light signals prior to the illuminating, using a modulation waveform having a first frequency;
demodulating the reflected signals; and
converting the demodulated reflected signals to an intermediate frequency prior to the processing.

5. The method of claim 1, further comprising:
prior to applying the artificial intelligence system, filling gaps in the absorption and scattering estimates.

6. The method of claim 1, wherein the target area contains non-cancerous tissue and the illuminating includes illuminating both the non-cancerous tissue and the suspected or confirmed cancerous tissue.

7. The method of claim 6, further comprising:
comparing absorption and scattering estimates from the non-cancerous tissue to corresponding absorption and scattering estimates from the suspected or confirmed cancerous tissue.

8. The method of claim 7, wherein the target area includes confirmed cancerous tissue, the method further comprising:
repeating the comparing using absorption and scattering estimates computed from a previous instance of illuminating to output a determination of whether the estimates from the confirmed cancerous tissue are converging towards the estimates from the non-cancerous tissue.

9. The method of claim 6, further comprising:
displaying an image of the target area together with the decision and the absorption and scattering estimates, wherein the image shows a region containing the non-cancerous tissue in a different format than a region containing the suspected or confirmed cancerous tissue.

10. The method of claim 1, further comprising:
receiving a confidence level for the decision from the artificial intelligence system, wherein the artificial intelligence system computes the confidence level based on previous absorption and scattering estimates.

11. The method of claim 1, further comprising:
receiving analysis results from the artificial intelligence system over the Internet, wherein the artificial intelligence system divides a task of determining whether cancer exists in the target area into a plurality of sub-tasks executed in parallel on cloud based computers.

12. The method of claim 11, wherein the artificial intelligence system is an expert system that coordinates the cloud based computers so that each cloud based computer first performs a non-exhaustive reading of material corresponding to respective subtasks and reports back the findings before determining whether to continue reading.

13. The method of claim 1, wherein the computer is a portable device that outputs the decision for display on a wirelessly connected smart phone or tablet computer.

14. The method of claim 1, further comprising:
applying the artificial intelligence system to make the decision based on data obtained from irradiating the target area using a second imaging technique.

15. The method of claim 14, wherein the second imaging technique includes at least one of irradiating the target area with acoustics and detecting acoustics emitted by the target area in response to irradiation.

16. A system for cancer detection using Diffuse Optical Spectroscopy, comprising:
a hardware interface that illuminates a target area containing suspected or confirmed cancerous tissue, using a plurality of light signals of different wavelength;
a parameter computation subsystem including a computer processor that processes corresponding reflected signals from the target area to compute estimates of absorption and scattering; and
an artificial intelligence subsystem that analyzes the absorption and scattering estimates to output a decision as to whether cancer is detected in the target area.

17. The system of claim 16, wherein the artificial intelligence subsystem includes at least one of a neural network and an expert system.

18. The system of claim 16, wherein the interface transmits the light signals to the target area through at least one transmission cable and using time division multiplexing, time division switching or wavelength division multiplexing, wherein the number of transmission cables is less than the number of light signals.

19. The system of claim 16, further comprising:
an optical-electronic subsystem that:
amplitude modulates the light signals prior to the illuminating, using a modulation waveform having a first frequency;
demodulates the reflected signals; and
converts the demodulated reflected signals to an intermediate frequency prior to the processing.

20. The system of claim 16, wherein the parameter computation subsystem fills gaps in the absorption and scattering estimates prior to the analysis by the artificial intelligence subsystem.

21. The system of claim 16, wherein the target area contains non-cancerous tissue and the illuminating includes illuminating both the non-cancerous tissue and the suspected or confirmed cancerous tissue.

22. The system of claim 21, wherein the artificial intelligence subsystem compares absorption and scattering estimates from the non-cancerous tissue to corresponding absorption and scattering estimates from the suspected or confirmed cancerous tissue.

23. The system of claim 22, wherein the target area includes confirmed cancerous tissue, and wherein the artificial intelligence subsystem repeats the comparing using absorption and scattering estimates computed from a previous instance of illuminating to output a determination of whether the estimates from the confirmed cancerous tissue are converging towards the estimates from the non-cancerous tissue.

24. The system of claim 21, further comprising:
a presentation subsystem that displays an image of the target area together with the decision and the absorption and scattering estimates, wherein the image shows a region containing the non-cancerous tissue in a different format than a region containing the suspected or confirmed cancerous tissue.

25. The system of claim 16, wherein the artificial intelligence subsystem computes a confidence level for the decision based on previous absorption and scattering estimates.

26. The system of claim 16, wherein the artificial intelligence system sends analysis results to the computer over the Internet, and wherein the artificial intelligence system divides a task of determining whether cancer exists in the target area into a plurality of sub-tasks executed in parallel on cloud based computers.

27. The system of claim 26, wherein the artificial intelligence system is an expert system that coordinates the cloud based computers so that each of the cloud based computers first performs a non-exhaustive reading of material corresponding to respective subtasks and reports back the findings before determining whether to continue reading.

28. The system of claim 16, further comprising:
a smart phone or tablet computer configured to display the decision, wherein the computer is a portable device that wirelessly outputs the decision to the smart phone or tablet computer.

29. The system of claim 16, wherein the artificial intelligence system makes the decision by analyzing data obtained from irradiating the target area using a second imaging technique.

30. The system of claim 29, wherein the second imaging technique includes at least one of irradiating the target area with acoustics and detecting acoustics emitted by the target area in response to irradiation.
